(19) United States
(12) Patent Application Publication
(10) Pub. No.: US 2019/0069836 A1
(43) Pub. Date: Mar. 7, 2019

(54) DETECTING EARLY TISSUE DAMAGE RESULTING FROM MECHANICAL DEFORMATION, SHEAR, FRICTION, AND/OR PROLONGED APPLICATION OF PRESSURE

(71) Applicant: NOVA SOUTHEASTERN UNIVERSITY, Fort Lauderdale, FL (US)

(72) Inventor: Heather HETTRICK, Fort Lauderdale, FL (US)

(73) Assignee: NOVA SOUTHEASTERN UNIVERSITY, Fort Lauderdale, FL (US)

(21) Appl. No.: 15/757,034

(22) PCT Filed: Aug. 30, 2016
(86) PCT No.: PCT/US16/49412

§ 371 Date: Mar. 2, 2018

Related U.S. Application Data
(60) Provisional application No. 62/214,960, filed on Sep. 5, 2015.

(51) Int. Cl. A61B 5/00; A61B 5/107; A61B 90/00; G01N 33/483

(52) U.S. Cl. CPC A61B 5/447; A61B 5/0077; A61B 2090/3937; A61B 90/39; G01N 33/4833; A61B 5/1079

(57) ABSTRACT

A system and method for using a forensic alternative light source (ALS) to detect tissue damage related to pressure ulcer/injury pathophysiology before visible manifestations of the tissue damage are evident with the naked (unaided) eye.

### CROSS REFERENCE TO RELATED APPLICATIONS

This application claims the benefit of priority to U.S. Provisional Patent Application No. 62/214,960 (filed on Sep. 5, 2015) the contents of which are hereby incorporated by reference.

### FIELD OF THE INVENTION

The invention generally relates to systems and methods for detecting tissue damage resulting from mechanical forces; particularly to early intervention in pressure ulcer/injury pathophysiology; and most particularly to a system and method for using a forensic alternative light source (ALS) to detect tissue injury related to pressure ulcer/injury pathophysiology before visible manifestations of the tissue injury are evident with the naked (unaided) eye.

### BACKGROUND

Pressure ulcers/injuries are an international problem, affecting millions of individuals. Incidence and prevalence data reveal the devastating toll this diagnosis has on health, quality of life, and health care costs. In the United States (U.S.) alone, pressure ulcers/injuries affect over 2.5 million people, costing in excess of $11 billion per year. Furthermore, over 60,000 deaths and 17,000 lawsuits annually are related to pressure ulcers/injuries.

Pressure ulcers/injuries are a huge burden on the health care system and significantly impact quality of life. The Centers for Medicare and Medicaid spent nearly $11 billion dollars in 2007 on full thickness pressure ulcers/injuries (Stages III, IV, and unstageable and deep tissue injury) reported in hospital settings. At any given time, nearly 15% (overall prevalence) of the U.S. population suffers from pressure ulcers/injuries. Elderly individuals are at the greatest risk for development of these wounds. For example, the incidence of pressure ulcers/injuries in long term care has been reported to be nearly 40%.

Pressure ulcers or injuries are defined by the National Pressure Ulcer Advisory Panel (NPUAP) as “localized damage to the skin and/or underlying soft tissue usually over a bony prominence or related to a medical or other device. The injury can present as intact skin or an open ulcer and may be painful. The injury occurs as a result of intense and/or prolonged pressure or pressure in combination with shear. The tolerance of soft tissue for pressure and shear may also be affected by microclimate, nutrition, perfusion, co-morbidities and condition of the soft tissue.” Information regarding NPUAP Staging Systems and Definitions is available on the NPUAP web page. Pressure ulcers/injuries are a common condition affecting all clinical settings and represent a costly cycle of recurrent hospitalizations, surgeries, office visits, and home care needs (Kruger, E. et al. J Spinal Cord Med. 36(6):572-585 2013). According to the US Joint Commission on Patient Safety, more than 2.5 million patients in acute care suffer from pressure ulcers/injuries, and over 60,000 die due to pressure ulcer related complications each year (Russo, C et al. Healthcare Cost Utilization Project December 2008, Statistical Brief #64). Estimates of pressure ulcer prevalence range from 10 to 18% in acute care, 2.3 to 28% in long term care and 0 to 29% in home care (Salcido, R. et al. Pressure Ulcers and Wound Care 1984, available at the Medscape web page). According to the Agency for Healthcare Research and Quality, pressure ulcers/injuries cost $9.1 to $11.6 billion per year in the US, with individual patient care costs ranging from $20,900 to $151,700 per pressure ulcer/injury. Medicare estimated in 2007 that each pressure ulcer/injury added $43,180 in costs to a hospital stay. Facts regarding pressure ulcers/injuries are available at the web page of the Agency for Healthcare Research and Quality web page.

Pressure ulcers/injuries are staged according to the depth of tissue injury. The first visible sign of tissue trauma associated with pressure and shear, is non-blanching erythema detected visually. Although detected at the surface, it is important to note that tissue damage has ensued due to ischemic and hypoxic changes in the tissue layers. The National Pressure Ulcer Advisory Panel (NPUAP) has established a staging system for pressure ulcers/injuries including: Stage I, II, III, IV, unstageable and deep tissue injury. Stage I is intact skin with non-blanching erythema. Stage II pressure ulcers/injuries are considered partial thickness and Stages III, IV, and unstageable and deep tissue injury are all considered full thickness ulcers of varying degree.

Pressure ulcers/injuries that develop on the heel are a particular challenge given the anatomy of the foot and the difficulty in keeping the feet offloaded. Unfortunately, approximately 36% of all pressure ulcers/injuries occur at the heel leading to limitations in mobility, possible infection and/or amputation of the foot, leg, and/or portions thereof.

Early detection of pressure ulcers/injuries is vital because of the socio-economic burden placed upon the health care system, the difficulty treating said ulcers/injuries in later stages, and the importance of skin integrity and its relation to function, mobility, and quality of life for patients. Currently, there are very few clinically useful tools to assist with early pressure ulcer/injury detection and prevention. As far as prevention strategies for pressure ulcers/injuries, the standard of care involves the use of a risk assessment tool to identify people at higher risk for developing ulcers/injuries in conjunction with interventions for prevention. However, there is no reliable evidence to suggest that the use of structured, systematic pressure ulcer/injury risk assessment tools reduces the incidence of pressure ulcers (Moore, Z. et al. Cochrane Database of Systemic Reviews 2014).

There is a critical and apparent need to develop new and valid clinical tools for pressure ulcer prevention and early detection. Current prevention interventions include repositioning, skin care (lotions, dressings, management of incontinence), nutritional support, and support surfaces for pressure redistribution (mattresses, overlays, cushions, integrated bed systems) (Moore, Z. et al. Cochrane Database of Systemic Reviews 2014).

Given the interventions just listed, there are no standardized or recommended early detection devices for pressure ulcers/injuries.

Multiple investigational detection devices have been studied with various results. There is research involving monitoring oxygen saturation in the skin, functional infrared imaging, enhanced imaging, multi-wavelength imaging, tissue reflectance spectroscopy, and even thermal imaging. However, no technique has been proven to be superior.

Forensic science has routinely used ultraviolet (UV) and infrared (IR) as alternative light sources (ALSs) to collect evidence such as latent finger prints, body fluids, hair, fibers and soft tissue injuries. More recently, ALS has been employed to detect intradermal bruising and strangulation injuries (Holbrook, D. et al. Journal of Forensic Nursing 9(3): 140-145 2013). ALS consists of a powerful light source that emits ultraviolet, visible, and infrared wavelengths. “ALS filters the light into individual color bands (wavelengths) that enhance the visualization of evidence by light interaction techniques: fluorescence (evidence that glows), absorption (evidence that darkens), and oblique lighting (small particle evidence that is revealed).” (Holbrook, D. et al. Journal of Forensic Nursing 9(3): 140-145 2013). In soft tissue injuries, under ALS, blood presents as evidence that darkens (absorption). The visible portion of the electromagnetic spectrum extends from ultraviolet wavelengths (190-400 nm), visible wavelengths (400-700 nm) to infrared wavelengths >700 nm. These light sources can reveal details in the skin that are invisible under normal white-light illumination. When attempting to detect soft tissue injuries, multiple wavelengths are necessary because different colors penetrate to different depths in the skin.

One aspect of the invention relates to the use of ALS to detect tissue trauma related to pressure ulcer/injury pathophysiology before visible manifestations of tissue injury are evident with the naked eye. Utilization and implementation of ALS to detect tissue trauma related to pressure ulcer/injury formation has the potential to provide a simple, non-invasive, clinically applicable tool for the detection and prevention of pressure ulcers/injuries in the medical field. The development of a valid prevention tool is vital for not only preventative measures, but to also understand the clinical course of pressure ulcers/injuries and intervention outcomes, and further refine health care policies to improve standards of care.

### SUMMARY OF THE INVENTION

Pressure ulcers/injuries, sometimes referred to as “bed sores” or “decubitus ulcers”, are an international problem affecting millions of individuals and costing billions of dollars. These ulcers/injuries are largely preventable and thus early detection in ulcer/injury development is essential for effective intervention and management. The instant inventor looked to forensic science for tools that are potentially useful in this endeavor.

Light in particular wavelengths is used at crime scenes and in forensics to detect blood and other body fluids, and to detect bite marks or bruises that otherwise cannot easily be seen. For example, a bruise pattern on the skin can indicate use of a particular weapon. Multiple wavelengths are used to penetrate to different depths within the skin, with deep wounds requiring infrared illumination in order to obtain details of crime-related bruises, caused, for example, by a bite or blunt force trauma. The instant inventor recognized that such methods of using light might likewise be applicable for detection of pressure ulcers/injuries.

The following descriptions of systems and methods are examples of embodiments of the instant invention and do not and should not be construed as representative of the entire scope.

The term “about”, as used herein, refers to near or close to the disclosed quantities.

In a broad aspect, the invention utilizes various wavelengths of light for detection of tissue changes before the changes are visible to the unaided eye.

In one aspect, the invention provides a system or a collection of devices useful in carrying out the methods described herein. One of these systems is a system for detecting changes in tissue indicative of tissue damage prior to visibility of the changes on skin with an unaided eye. The phrases “naked eye” and “unaided eye” are used interchangeably herein to describe the action of observing with the bodily eye having normal vision alone in the absence of outward assistance such as, but not limited to, glasses, contact lens, microscopes, and/or invisible wavelengths of light akin to those exemplified in the methods described herein. This system can be used to detect any type of change or alteration in tissue, particularly those changes indicative of damaged and/or unhealthy tissue. The system is most particularly useful for detecting tissue changes/damage resulting from mechanical force applied over time, such as, but not limited to, mechanical deformation, pressure, shear, and friction. General components of the system include, but are not limited to, an alternative light source (ALS), a plurality of filters for a camera, and a camera.

The alternative light source (ALS) of the described system is configured for emitting light in one or all of the ultraviolet, visible, and infrared wavelengths and is used for illuminating an area of tissue to be evaluated for changes. A preferred ALS is a forensic alternative light source (ALS) which is portable, handheld, and thus easily transported and used. A specific non-limiting example of a forensic ALS that is preferentially utilized when carrying out the described methods is the SPEX Forensics Mini-CrimeScope®. The SPEX Forensics Mini-CrimeScope® has a wide range of function and is usable at and switchable between 6, 8, 12, or 16 wavelengths of light. Examples of wavelengths of light produced are frequencies of 365 nm (ultraviolet), 390 nm, 415 nm, 445 nm, 455 nm, 475 nm, 495 nm, CSS SP (Short Pass) 540 nm, 515 nm, 535 nm, 555 nm, SP 575 nm, 575 nm, 600 nm, and white light.

The camera of the described system is configured for obtaining images of the area of tissue illuminated with the alternative light source (ALS) and can be any camera deemed appropriate for carrying out the described methods. A specific non-limiting example of a camera that is preferentially utilized when carrying out the described methods is an SLR (single-lens reflex) camera. The camera can be used separately from the ALS or can be affixed to the ALS. A tripod for supporting the camera can also be included as a component of the system.

Each of the plurality of filters for the camera is configured for obtaining images using a different wavelength of light emitted from the alternative light source (ALS). The plurality of filters include, but are not limited to, a red camera lens, a yellow camera lens, and an orange camera lens.

In another aspect of the invention, the system contains additional components useful in carrying out the methods described herein.

An example of additional components is a plurality of goggles/lenses that are useable with the plurality of filters for the camera. Different colored goggles are used with different wavelengths/filters under ALS illumination to detect the presence or absence of tissue changes and/or damage. Preferential colors of goggles included in the system are red goggles, yellow goggles, orange goggles, and black out goggles.

Another additional component is a photographic grid useful for data collection; i.e. measurement and evaluation of the images obtained by the camera.

Further additional components include one or more black sheets useful for providing contrast to the images and for reducing natural light. For example, a black sheet can be placed near to or underneath an area of tissue to be evaluated and/or could be used to cover any windows in the test area to reduce natural light.

In another broad aspect, the invention provides methods of using a forensic alternative light source (ALS) to detect tissue changes before the changes are visible to the unaided eye.

One embodiment representative of the inventive methods is a method for detecting tissue damage due to at least one of mechanical deformation, pressure, shear, and friction applied to skin over time. This method includes general steps for illuminating skin using a forensic alternative light source (ALS) configured for examining crime evidence using a frequency of light selected for revealing tissue damage; and observing the skin for indications of tissue damage visible only when the selected frequency of light is used. Any tissue damage present will absorb the light. The systems and ALS devices described above can be used to carry out this method.

An aspect of this method includes selecting an area of skin for illuminating. Tissue changes and/or damage resulting from mechanical force, i.e. mechanical deformation, shear, pressure, and friction applied to skin over time, can occur on any area of the body but are most likely to occur at a bony prominence; i.e. an area of skin overlying bone. Several exemplary non-limiting examples of these bony prominences are an area of skin overlying a heel, a pelvis, a shoulder, a spine, a wrist, or an elbow of the patient.

Another aspect of this method includes selecting the frequency of light at which to set the ALS for the illuminating. Several exemplary non-limiting frequencies are a violet wavelength at about 415-445 nm, a blue wavelength at about 455-515 nm, and a green wavelength at about 535-575 nm. Upon selecting the frequency selecting a lens specific for the selected wavelengths of light and viewing the illuminated skin with the selected lens. Several exemplary non-limiting wavelengths are a violet wavelength using a yellow camera lens, a blue wavelength using an orange camera lens, and a green wavelength using a red camera lens.

Another aspect of this method includes obtaining at least one image of the area of skin illuminated. Obtaining images includes obtaining a single image, a series of images, and/or a series of images over a range of time. Additionally, it is preferred, but not required to obtain at least one image of the area of skin to be illuminated with ambient light prior to illuminating with the forensic alternative light source (ALS) to establish a baseline for comparison with the illuminated images.

Another embodiment representative of the inventive methods is a method for detecting changes in tissue indicative of tissue damage prior to visibility of the changes on skin with an unaided eye. This method can be used to detect any type of change or alteration in tissue, particularly those changes indicative of damaged and/or unhealthy tissue. The method is most particularly useful for detecting tissue changes/damage resulting from mechanical force applied over time, such as, but not limited to, mechanical deformation, pressure, shear, and friction. This method is preferentially carried out with a human subject but is not limited thereto. General steps of this method include selecting an area of skin of a subject to be evaluated for changes; optimizing position of the subject for data collection; establishing a baseline for the data collection by obtaining at least one image of the area of skin in ambient light using a camera; reducing ambient light; illuminating the area of skin using an alternative light source (ALS) configured for emitting light in one or all of the ultraviolet, visible, and infrared wavelengths; obtaining a series of images of the area of skin illuminated by different wavelengths of light using the camera; evaluating the images for changes in the tissue; and documenting any changes observed in the tissue.

An aspect of optimizing position of the subject includes, but is not limited to, having the subject lie on his/her side.

Another aspect of this method includes pre-starting the alternative light source (ALS) prior to illuminating the area of skin. This pre-starting allows for maximum luminosity of the ALS.

Another aspect of this method includes selecting the frequency of light at which to set the ALS for the illuminating. Several exemplary non-limiting frequencies are a violet wavelength at about 415-445 nm, a blue wavelength at about 455-515 nm, and a green wavelength at about 535-575 nm. Upon selecting the frequency selecting a lens specific for the selected wavelengths of light and viewing the illuminated skin with the selected lens. Several exemplary non-limiting wavelengths are a violet wavelength using a yellow camera lens, a blue wavelength using an orange camera lens, and a green wavelength using a red camera lens.

Another aspect of this method includes using goggles to view the illuminated areas of skin. There are a plurality of goggles/lenses that are useable with the plurality of filters for the camera. Different colored goggles are used with different wavelengths/filters under ALS illumination to detect the presence or absence of tissue changes and/or damage. Preferential colors of goggles included in the system are red goggles, yellow goggles, orange googles, and black out goggles.

Multiple combinations of wavelengths, lens, and goggles could be employed with this method to view the illuminated areas of skin. Several non-limiting examples are viewing a violet spectrum at a wavelength of 415 nm and a wavelength of 445 nm using a yellow camera lens and yellow goggles, viewing a blue spectrum at a wavelengths of 455 nm, 475 nm, 495 nm, and 515 nm using an orange camera lens and orange goggles, and viewing a green spectrum at wavelengths of 535 nm, 555 nm, and 575 nm using a red camera lens and red goggles. One preferred example is viewing the blue spectrum at the wavelength of 455 nm and at the wavelength of 475 nm using the yellow camera lens and the yellow goggles.

Another aspect of this method includes selecting an area of skin overlying bone for illuminating. Tissue changes and/or damage resulting from mechanical force, i.e. mechanical deformation, pressure, shear, and friction applied to skin over time, can occur on any area of the body but are most likely to occur at a bony prominence; i.e. an area of skin overlying bone. Several exemplary non-limiting examples of these bony prominences are an area of skin overlying a heel, a pelvis, a shoulder, a spine, a wrist, or an elbow of the patient. A particularly preferred, but non-limiting, example is an area of skin overlying a heel of the subject.

Another aspect of this method includes obtaining a series of images of the area of skin illuminated by different wavelengths of light using the camera. A series of images may include any number of images beyond a single image. A preferred, but non-limiting, example is series of photos having a minimum of 12 photos/images of each subject.

Another aspect of this method includes optimizing the parameters of the camera prior to use. Any suitable parameters could be set and used. Preferred, but non-limiting parameters include setting the F stop of the camera between 2.8 to 8 and setting the exposure time of the camera at 1/100 of a second.

Another aspect of this method includes optimizing the positioning of the camera prior to use. Any position effective to obtain images could be used. A preferred, but non-limiting, example is placing the camera at a distance in a range of about 6 inches (15.24 cm) to about 24 inches (60.96 cm) from the subject. A particularly preferred example is placing the camera at a distance of at least 24 inches (60.96 cm) from the subject.

Another aspect of this method includes evaluating the images for changes in the tissue. Any effective evaluation can be used. Preferred, but non-limiting, evaluations include determining an amount of light absorbed by the tissue and determining a relationship between the wavelength used and a number of tissue injuries detected.

Another aspect of this method includes repeating the method for all subjects over a pre-determined period of time. Any suitable pre-determined period of time can be used. A preferred, but not limiting, example is repeating the method once a week for 6 consecutive weeks. The present invention contemplates that the method can be repeated as desired, or when there is a concern about the subject's skin condition, or any time the subject's clinical condition changes.

Any of the systems described herein can be used in any of the methods described herein. For example, the described system can be used in detecting changes in tissue indicative of tissue damage prior to visibility of the changes on skin with an unaided eye.

Other objectives and advantages of this invention will become apparent from the following description taken in conjunction with the accompanying drawings, wherein are set forth, by way of illustration and example, certain embodiments of this invention. The drawings constitute a part of this specification and include exemplary embodiments of the present invention and illustrate various objects and features thereof.

### BRIEF DESCRIPTION OF THE DRAWINGS

A more complete understanding of the present invention may be obtained by references to the accompanying drawings when considered in conjunction with the subsequent detailed description. The embodiments illustrated in the drawings are intended only to exemplify the invention and should not be construed as limiting the invention to the illustrated embodiments.

FIG. 1 is a photograph of an arm of a patient taken while the arm is illuminated in normal room lighting (ambient) light.

FIG. 2 is a photograph of the arm of FIG. 1, taken while the arm is illuminated using a forensic alternative light source (ALS), showing acute bruising which was not visible in FIG. 1.

FIG. 3 shows a forensic ALS device useable to detect and treat incipient tissue damage in accordance with the instant disclosure.

FIG. 4 shows a view of a handheld configurable handpiece of the device of FIG. 3.

FIG. 5 shows a front view of the handpiece of FIG. 3.

FIG. 6 shows a perspective view of lenses (goggles) that are usable with the device of FIG. 3. The shading represents different colors so that each lens or goggle filters out different wavelengths of light.

FIG. 7 shows a camera and filter useable with the ALS.

FIG. 8 is a cluster dendogram showing the correlation of the images.

FIG. 9 is a photograph of a heel of a patient (Stage I) taken while the heel is illuminated in normal room lighting (ambient) light.

FIG. 10 is a photograph of the heel of the patient (Stage I) shown in FIG. 9 taken while the heel is illuminated using the ALS at a wavelength of 415 nm.

FIG. 11 is a photograph of the heel of the patient (Stage I) shown in FIG. 9 taken while the heel is illuminated using the ALS at a wavelength of 475 nm.

FIG. 12 is a photograph of a heel of a patient (unstageable) taken while the heel is illuminated in normal room lighting (ambient) light.

FIG. 13 is a photograph of the heel of the patient (unstageable) shown in FIG. 12 taken while the heel is illuminated using the ALS at a wavelength of 445 nm.

FIG. 14 is a photograph of the heel of a patient (unstageable) taken while the heel is illuminated in normal room lighting (ambient) light. This photograph was taken a week after the photograph of FIG. 12 was taken.

FIG. 15 is a photograph of the heel of the patient (unstageable) shown in FIG. 14 taken while the heel is illuminated using the ALS at a wavelength of 475 nm.

FIG. 16 is a photograph of a heel of a patient (scars) taken while the heel is illuminated in normal room lighting (ambient) light.

FIG. 17 is a photograph of the heel of the patient (scars) shown in FIG. 16 taken while the heel is illuminated using the ALS at a wavelength of 475 nm.

### DETAILED DESCRIPTION OF THE INVENTION

For the purpose of promoting an understanding of the principles of the invention, reference will now be made to embodiments illustrated herein and specific language will be used to describe the same. It will nevertheless be understood that no limitation of the scope of the invention is thereby intended. Any alterations and further modification in the described systems and methods and any further application of the principles of the invention as described herein, are contemplated as would normally occur to one skilled in the art to which the invention relates.

The invention provides, inter alia, in one aspect, a method for detecting tissue damage due to mechanical deformation, shear, friction, and/or prolonged application of pressure, before the onset of excessive damage or pressure injury is evident. Such early detection of tissue damage is carried out using a forensic alternate light source device (ALS), which is used to illuminate the skin of a patient at risk for pressure ulcers/injury. The forensic alternate light source (ALS) device illuminates tissue and causes damaged tissue to absorb the light, whereby tissue destruction can be detected and identified at an early stage, before it becomes sufficiently severe to be visible to the unaided eye. More particularly, the forensic light source reveals bruise and wound details that are invisible under normal white light illumination such as is provided by sunlight, or typical incandescent or fluorescent lighting. The forensic light source device can be used with a camera and colored lenses to capture any absorption detected by the ALS, so that historical data can be referenced and/or shared with other health practitioners.

Another aspect of the method of the disclosed invention uses forensic light to provide for a non-invasive approach to early intervention of pressure ulcers/injuries, resulting in improving the health and quality of life for patients, and saving substantial resources spent on treating pressure ulcers/injuries. Thus, within the disclosure, forensic related technology is applied in the area of health science, including wound prevention and management, early identification and intervention, and in particular to the prevention and management of pressure ulcers/injuries, by detecting tissue damage prior to visible tissue destruction.

Despite advances in pressure ulcer/injury prevention through risk assessment, pressure-redistribution surfaces, and evidence-based interventions, pressure ulcers/injuries still remain a leading health care concern worldwide; particularly when many pressure ulcers/injuries can be prevented if detected sufficiently early. Pressure ulcers/injuries typically initially manifest at the bony tissue interface given bone and deep tissue structures have less resistance to hypoxia and ischemic changes compared to the skin. Skin has the highest resistance to hypoxia and therefore, has a more latent response to damage compared to the tissue structures beneath. In accordance with the disclosure, these tissue changes begin to occur prior to visual and physical manifestations on the skin which can be perceived by unaided visual inspection. A forensic light source has been found to reveal these tissue changes prior to a time when they are visible by the human eye under normal light conditions.

Prior to the methods of the disclosure, health practitioners needed to rely on risk reduction strategies to prevent pressure ulcers/injuries, as no noninvasive tools have been available for detecting pressure trauma before physical and visual signs occur. In the disclosure, an alternative light source (ALS), or forensic light, is used to reveal bruises, tissue damage, and patterned wound details that are invisible under normal white light illumination, but which have been identified to correlate with an early indication of tissue damage due to mechanical deformation, pressure, friction, and/or shear. Various light wavelengths are generated by the forensic light, and each can be used to enable light penetration to a different depth within the skin. Deep wounds, such as those caused by pressure, can be revealed using infrared illumination. Other wavelengths between and including infrared and ultraviolet wavelengths can be used to reveal damage at particular skin depths, and at depths closer to the skin surface.

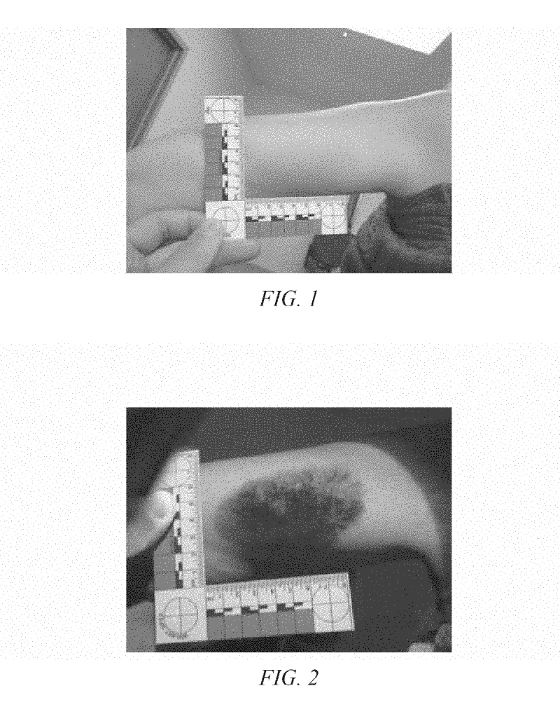

Examples of visualization of tissue damage are shown in FIGS. 1 and 2. FIG. 1 shows an arm of a patient in natural white light, with a measuring grid provided for orientation and standardization. In FIG. 2, the arm of the same patent is being illuminated by the use of a forensic ALS as described herein, showing evidence of tissue damage which is not visible in FIG. 1. The tissue damage is noted by the absorption of light. Thus, the invention provides a means of detecting tissue damage, as shown, which was undetectable by the unaided eye using white light or normal room or outdoor lighting.

Further in accordance with the invention, a method for early detection of pressure ulcers/injuries or incipient pressure ulcer formation includes evaluating the skin of patients who are in bed for extended periods, or who otherwise are subjecting skin to prolonged contact with an object. Bony prominences are most at risk for pressure ulcer/injury formation; typically this includes the upper and lower back, hip, buttock, ankle, heel, elbow, and shoulder. Pressure ulcers/injuries can occur at other places as well, such as in any place where excessive or prolonged pressure is applied. If sufficiently responsive, the patient can indicate skin areas which are experiencing discomfort, and which should be evaluated. However, in many situations, patients may not be responsive and therefore unable to indicate if or where a problem is occurring.

Patients can be selected for evaluation or screening of incipient pressure ulcers/injuries or pressure induced tissue damage, in accordance with the invention, based on known risks or profiles, which reflect observed incidences of pressure ulcers in various populations. For example, the incidence of pressure ulcers/injuries in long term care has been reported to be nearly 40%, while the overall prevalence in the U.S. is approximately 15%. Elderly individuals are at a greater risk for the development of pressure ulcers/injuries, generally, than younger people. High risk patients can be evaluated weekly or daily, depending upon their risk profile. As daily assessments are performed on all residents in a nursing home, screening according to the disclosure can be carried out without significantly changing their preexisting daily routines, plan of care, positioning, or skin interventions.

For certain wavelengths, and due to the relatively high strength of the forensic light (luminosity), goggles 5-8 (as shown in FIG. 6; red goggles 5, orange goggles 6, yellow goggles 7, and clear goggles 8) can be provided for the patient as well as the health practitioners carrying out the screening procedure. Black out goggles, which do not allow the passage of any light of any wavelength can be used for maximum protection. Different colored goggles are used with different wavelengths under ALS illumination to detect the presence or absence of tissue damage. For example, absorption or a darkening in the tissues is indicative of blood in the tissues due to tissue damage. For deep tissue screening, infrared light may be used. However, other wavelengths can also be used to detect changes to the skin and beneath the skin.

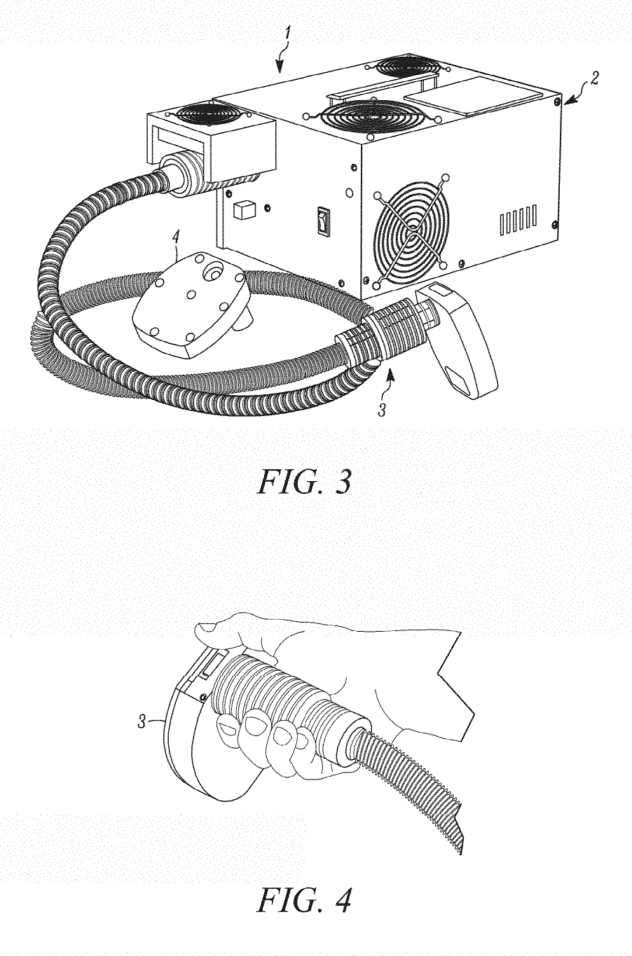

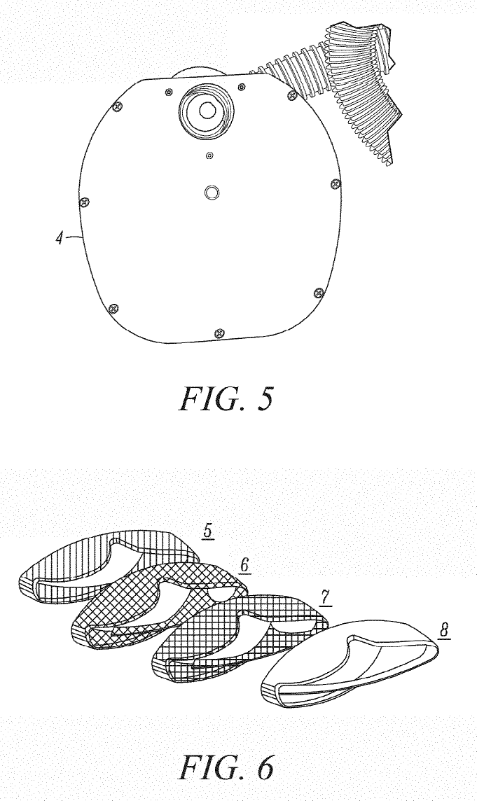

A preferred, albeit non-limiting, example of a forensic alternative light source (ALS) device is shown in FIGS. 3-5; a SPEX Forensics Mini-CrimeScope®. This device is manufactured by SPEX Forensics of New Jersey. The device 1 includes a body 2 encasing a 400W metal halide arc lamp, producing illumination which is passed through a flexible 6 foot light guide to a handheld lens 3 (FIGS. 3 and 4) which includes a detachable selectable light filter 4 (FIGS. 3 and 5). Details of aspects of the handheld device can be found in U.S. Pat. No. 6,862,093. The device is switchable among 6, 8, 12, or 16 wavelengths, selected among these frequencies, in nanometers: 365(UV), 390, 415, 445, 455, 475, 495, CSS SP (Short Pass) 540, 515, 535, 555, SP 575, 575, 600, and WHITE.

A forensic ALS includes a powerful light containing one or all of the ultraviolet, visible, and infrared components of the Electromagnetic Spectrum. This light is a filter where individual color bands (wavelengths) are selected that enhance the visualization of evidence by light interaction techniques. Currently, forensic light sources are used to examine fingerprints—latent and superglue, body fluids, trace evidence, bruising, bite marks, bone fragments, questioned documents, and gunshot residue.

Forensic ALS devices can reveal bruise and wound details that are invisible under normal white light illumination. Multiple wavelengths are advantageous, as different colors penetrate to different depths within the skin. Deep wounds often require infrared illumination in order to obtain sufficient skin penetration for visualizing a full extent of tissue involvement. Accordingly, higher power (energy) can result in better results, although it should be understood that the forensic ALS in accordance with the invention is to be used with living patients, and must not expose the potentially damaged skin area of the patient to a painful or harmful amount of light. The invention does not, however, expose the skin for long periods, and thus the forensic ALS units tested have not been harmful.

Forensic ALS devices can illuminate target evidence in the following ways: Fluorescence causes the evidence to glow; Absorption causes the evidence to darken; and Oblique lighting causes small particle evidence to be revealed. When light strikes a surface or compound, it will either be absorbed, reflected, transmitted, or a combination of all 3. The actual interaction is between the photon of light and electrons bound to the atoms of the surface. Reflection occurs when the free electrons do not permanently absorb the incoming light, but release the light almost immediately. Scatter is a special case of reflection. More particularly, a rough object can also reflect light, but because of its non-smooth surface, the light is reflected in random directions. In the use of a forensic ALS, dust particles, hairs, fibers, and imprints of dust all scatter light extensively.

A wavelength is selected to best cause a target material to fluoresce in contrast to a background, to illuminate a particular target crime evidence, including fingerprints (on porous/non porous surfaces), body fluids, bite marks and bruises, shoeprints, gunshot residues, bone fragments, and drugs. Example light output includes the following: White Light Beam Output: Average: 14,000 lux, Minimum: 12,000 lux; 535 nm Light Beam Output: Average: 4,500 lux, Minimum: 3,500 lux. The device weight is 15 pounds, enabling it to be easily moved between patient rooms or locations.

Absorption occurs when light of a given wavelength is absorbed by a molecule's electrons, and thus the molecule appears darker than the surrounding environment. This absorbed light transfers its energy into the electrons of the molecule. Every-day colored objects, such as paint, cloth, human skin, and plastic, all absorb some wavelengths of light and reflect or transmit others. Without being bound to a particular theory, it is believed that tissue damage which has or may develop into a pressure ulcer/injury may damage deep tissue structures first, including bone, muscle, and tendons, before the skin is disrupted, given that skin has the highest resistance to hypoxia. Accordingly, it is expected that the damage from pressure and shear will cause microtrauma and myocutaneous infarction leading to tissue death and the release of blood products into the tissues. In accordance with the invention, the forensic ALS light in one or more wavelengths will detect this via, at least, absorption. It is noted that the depth of the skin involved is typically between 3 and 7 mm, and is therefore reachable at the greatest depth by, at least, infrared light, and to varying depths by the shorter wavelengths generated by the forensic ALS, as well.

The use of forensic ALS with particular wavelengths and filters is used in accordance with the invention to find and to detect pressure ulcers/injuries or other damage due to mechanical deformation, pressure, friction, and/or shear before the tissue damage becomes visible with the naked eye. The forensic ALS typically illuminates early tissue damage as a darkening, much like a bruise, whether dermally or subdermally, when the blood product and trauma of the early damage absorb the forensic ALS light, thus indicating damage.

To photograph tissue damage revealed by light from the forensic device, a camera can be used to obtain an image of the area illuminated by the selected wavelength, for the record, or for later evaluation. The camera, which can be a digital SLR or any other type of camera, can be affixed to the forensic device, or can be used separately. The camera can be equipped with a filter or lens that is selected to enhance contrast between the light source and body tissue. Accordingly, filters can be selected, for example, from deep yellow, orange, light red, and red, to correspond to the visible portion of the various wavelengths that are selected.

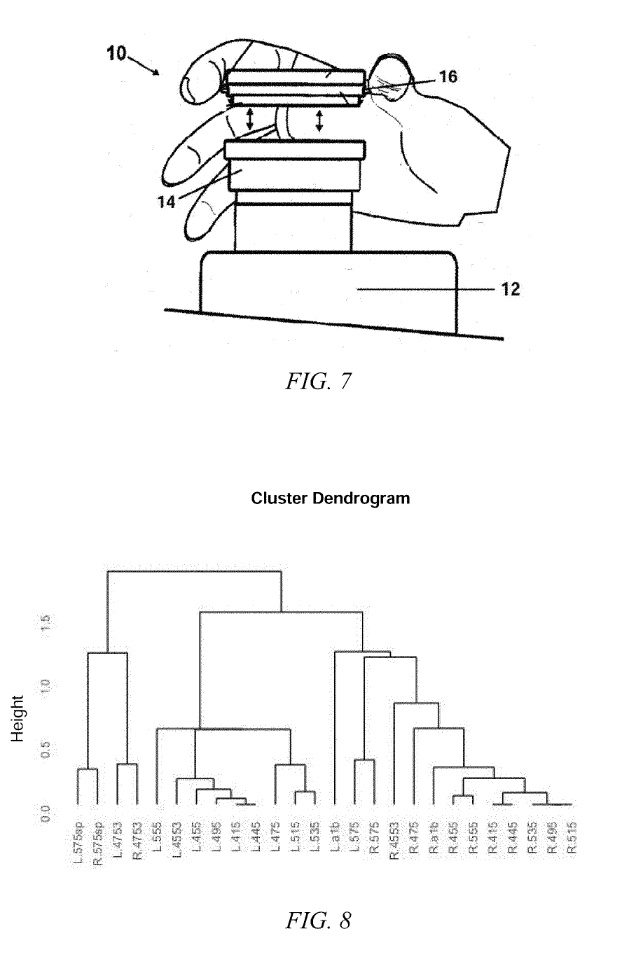

FIG. 7 shows an exemplary embodiment of a camera 10 that can be used as part of the current invention. Camera 10 includes a camera body 12 with lens 14. Lens 14 can be permanently attached or removably attached to camera body 12. Lens 14 can be a fixed or variable focus lens. A filter 16 can be permanently attached or removably attached to lens 14. As noted above, filter 16 is selected to filter out different wavelengths of light.

Heels represent 36% of all pressure ulcers/injuries, and can be evaluated without significantly disrupting the patient. Accordingly, a method of the invention for detecting incipient tissue damage uses heels as an example, although it should be understood that this example can be applied to any other area of skin on the body. Additionally, consent of the patient is advantageously obtained, although the methods of the invention are not expected to produce any bodily harm, provided the eyes of the patient and others in proximity are adequately protected from direct exposure to the light source.

Patients can be positioned lying on their sides in order to comfortably expose their heels. If side-lying is not possible, the patient can may remain in a supine position with the heels elevated. For other body parts, an appropriate and comfortable position of the patient is determined, which enables visualization of the skin area to be evaluated. With consent, an optional standard photograph of the subject's heels can be taken for the record and for later evaluation, followed by exposure of the area of interest, for example the back of the heel, to the forensic light. The light will reveal changes in underlying tissue, including ischemic and hypoxic changes in the tissue layers, such as are shown, for example, in FIG. 2.

In one embodiment, in order to be minimally disruptive to patients, screenings are conducted either early in the morning or evening while subjects may still be in bed. This improves the potential for a darkened room, which can enhance the ability to view the affected tissues, and to improve the quality of any photographs.

A small printed grid can be placed next to the heels or other skin area for orientation and photographic documentation. Lights in the room can be dimmed or turned off during use of the forensic ALS and photography. Further, the test area can be draped with black or dark cloth to improve visualization and photography, eliminating as much white light as possible.

In another embodiment, visualization is carried out using a range of wavelengths, in order to fully visualize tissue damage at varying skin depths, and which reflects different types of tissue damage. When conducting photography, the following wavelengths from the ALS can best be photographed with the corresponding camera lens filter color: violet wavelength at 415-445 nm with yellow lens; blue wavelength at 455-515 nm with orange lens; and green wavelength at 535-575 nm with red lens. These frequencies have been found to be advantageous for detecting the type of bruising and tissue damage associated with prolonged mechanical forces; deformation, pressure, friction, and/or shear. These frequencies have been found to be particularly advantageous, however useful observable results can be obtained when the frequency is varied by 10%, or even up to 20-40%.

In another embodiment, the forensic ALS and a camera, if used, are placed at a distance from the anticipated problem area, for example at a distance in a range of about 12 inches (30.48 cm) to about 18 inches (45.72 cm) from the skin. This distance can vary, for example, dependent upon ambient light, the strength of the ALS light, the thickness of the skin, the frequency used, and the depth of the incipient damage. Typically, the light can be held as close as about 6 inches (15.24 cm) or less, and as far away as several feet (90 cm) or more.

To ensure that treatment has been effective for detected tissue damage, it is advantageous to conduct follow up screenings, for example weekly. It is further advantageous to avoid disrupting the patient's daily routine, plan of care, or interventions, including positioning or offloading of patients. The screening can therefore be carried out in addition to existing assessments and interventions.

Thus, in accordance with the invention, forensic ALS is used to detect pressure ulcer/injury formation subdermally/dermally before the ulcers/injuries become visible on the skin. As ALS can detect impending or incipient pressure ulcers/injuries before they become visible with the naked eye, the methods of the invention can be used for detecting and preventing further tissue damage. The application of forensic ALS as described herein is therefore usable for pressure ulcer/injury detection, early identification and subsequent intervention for the benefit of the patient and those responsible for patient care. Intervention can include aggressive off-loading of the body part in question, combined with frequent repositioning and the possible use of specialized support surfaces to reduce pressure and shear.

### EXPERIMENTAL EXAMPLE

This study was undertaken to answer: Is a forensic alternate light source and camera a valid and reliable tool to noninvasively detect tissue damage related to mechanical forces before they become visible to the naked eye?

Summary of Findings: In this prospective, single-institution repeated measures study that included 7 subjects, the alternate light source (ALS) showed significant tissue absorption indicating the actual scope and magnitude of tissue trauma not visible to the naked eye. Descriptive statistics (Table 1) and chi-square tests of independence (Table 2) specified the relationship between wavelengths and number of detected injuries.

Relevance of the Findings: ALS can detect tissue trauma related to mechanical forces not visible to the naked eye that could help identify patients in the early stages of tissue trauma as well as screen for sites of previous injury that are at risk for subsequent breakdown. Clinical application of this tool to screen for tissue trauma not readily visible to the naked eye has the ability to improve outcomes and quality of life while significantly reducing health care costs.

Importance: In the United States, pressure ulcers/injuries affect over 2.5 million people, costing in excess of $11 billion per year. Over 60,000 deaths and 17,000 lawsuits are related to pressure ulcers/injuries annually. Despite these facts, there are few clinical tools available to aid in early detection of pressure ulcers/injuries.

Objective: To determine if a commercially available forensic alternate light source (ALS, SPEX Forensics Mini-CrimeScope® and camera) can be used as a valid and reliable noninvasive tool to detect tissue changes as a result of mechanical forces prior to visual manifestations on the skin. Design: Prospective, single-institution observational study with repeated measures. Setting: Long-term care facility in West Palm Beach, Fla.

Participants: Medically sound eligible subjects were referred by Director of Nursing. Ten subjects were recruited and consented, 7 completed the study.

Exposures: Researchers examined and photographed subjects' heels in ambient light to establish baseline. A series of photographs using the ALS were taken as follows: Violet wavelength at 415-445 nm with yellow lens; Blue wavelength at 455-515 nm with orange lens; Green wavelength at 535-575 nm with red lens.

Main Outcome(s) and Measure(s): Subjects were examined weekly for 6 consecutive weeks to ascertain skin changes in ambient light and through the ALS camera.

Results: Overt tissue changes were noted when viewed with the ALS compared to visual screens in ambient light. Descriptive statistics were calculated for all wavelengths (Table 1). Two chi-square tests of independence (Table 2) were run to look for relationships between wavelength and the number of detected injuries (absorption).

Conclusions and Relevance: Subjects presenting with non-blanching erythema in ambient light showed significant tissue absorption under ALS, depicting the actual scope and magnitude of the tissue trauma. Subjects with scars, areas of previous injury, and pigmentary changes also showed significant absorption at those sites. These combined findings indicate that ALS can detect tissue trauma and areas at risk not readily visible by the naked (unaided) eye. This non-invasive tool could help identify patients in the early stages of tissue trauma as well as screen for sites of previous injury that are at risk for subsequent breakdown, saving significant health care dollars and improving outcomes and quality of life.

Detail of the Methods

Institutional Review Board approval was obtained at Nova Southeastern University, Fort Lauderdale Fla. A prospective, single-institution, repeated-measures design was employed to collect subject data. The study was conducted at a long-term care facility in West Palm Beach Fla. Potential subjects were referred by the Director of Nursing to the Primary Investigator. Study participation was limited to residents who were medically stable with at least one intact lower extremity given the body region under investigation involved the heels. Ten subjects (5 men and 5 women) were consented by the PI and co-PI; 7 were able to self-consent and 3 were consented by healthcare proxy (children of the subjects). One subject (male) passed away before any data were gathered. Another subject (female) participated in data collection; however, the equipment malfunctioned and data was not able to be collected. This subject subsequently withdrew from the study, as she did not want continue participating in the study. Malfunctioning equipment was replaced and data collection began for the remaining subjects. Data were collected for 5 consecutive weeks with a follow up visit several weeks later to ascertain subject status. Seven subjects completed the study.

Study equipment included SPEX Forensics Mini-Crimescope®, SLR (single-lens reflex) camera, tripod, yellow, orange, and red camera lenses; yellow, orange, and red goggles; black out goggles (for protection of subject and research assistants), 2 black sheets, 2 foam rolls, white board with dry erase marker and a photographic grid. The study protocol involved positioning the subjects in bed, side-lying on their preferential side. Prior to data collection, the SPEX Forensics Mini-Crimescope® was plugged into a wall socket (pre-starting) and the light source turned on to warm up for maximum luminosity. The camera, tripod, lenses and goggles were arranged for easy access during data collection. Yellow, orange and red goggles were worn by the PI taking the photographs and Research Assistant 1 operating the light source to view the ALS findings. Yellow, orange and red lenses were used on the SLR camera to photograph the findings viewed with the ALS. The camera F stop (aperture) was set as low as possible to allow the proper light and ranged from 2.8 to 8. The exposure time was set at 1/100 of a second and camera distance from subject area was set at 24-inches. Data were collected in the evening to allow for minimal natural light and for subject convenience.

A black sheet was placed under the subjects' legs and the lower extremities were supported with foam rolls to position and provide comfort during data collection. One roll was placed beneath the lower leg to elevate above the bed surface, and the other was placed between the medial malleoli to separate the feet and heels to maximize viewing area. The second black sheet was used to provide a backdrop and to maximize contrast for the photographs. A white board and photographic grid was placed next to the subject's feet containing the following information included in each photograph: Side-lying position indicated by L or R (left side-lying or right side-lying), subject number, date, and wavelength in nanometers (nm).

Subject's heels were first photographed in ambient light. Next, a series of photographs were taken using the ALS and SLR camera. Window shades were drawn and room lights were turned off for the ALS data collection. The violet spectrum was viewed using 415 and 445 wavelengths with a yellow camera lens and yellow goggles. The blue spectrum was viewed using 455, 475, 495 and 515 wavelengths with an orange camera lens and orange goggles. The 455 and 475 wavelengths were also viewed under the yellow lens and goggle as this combination effectively showed absorption. The green spectrum was viewed using 535, 555 and 575 wavelengths with a red camera lens and red goggles. A minimum of 12 photos were taken per subject per week. If absorption was noted, it was documented on the data collection sheet by the respective wavelength.

The roles and responsibilities of the research team were as follows: the PI took photographs and managed the camera; the co-PI recorded findings on data the collection sheets; Research Assistant 1 handled the SPEX Forensics Mini-Crimescope® which provided the light source for data collection; Research Assistants 2 and 3, draped the second black sheet to provide contrast and supported/guarded the subject during data collection. The PI and Research Assistant 1 verified whether absorption was present or not. For each subject, it took roughly 30 minutes to complete data collection per visit. Additionally, the PI had full access to all of the data in the study and takes responsibility for the integrity of the data and the accuracy of the data analysis.

Detail of the Results

Seven subjects completed the study. The findings from the data collection sheets were transferred into an Excel spreadsheet for statistical analysis. Subject data were entered into the spreadsheet for each week data were collected. Findings were reported for each wavelength and noted as either “M” for Missing (photo not clear or unable to obtain), “Y” for Yes indicating absorption, or “N” for No indicating no change or no absorption.

Descriptive statistics were calculated for all wavelengths (Table 1). Two analyses were conducted to look for relationships between wavelength and detecting injury (absorption). For analysis one, different wavelengths were grouped into two categories: (1) 455 Y and 475 Y vs Other; and for analysis two the groups were extended: (2) 455, 455 Y, 475, 475 Y, 495, and 515 vs Other. To look for differences under both scenarios, two chi-square tests of independence (Table 2) were run to look for relationships between wavelength and the number of detected injuries (absorption). Results are as follows:
For analysis one, the percentage of findings did not differ by grouping, c² (2, N=1540)=2.71, p=0.257
For analysis two, the percentage of findings did differ by grouping, c² (2, N=1540)=11.95, p=0.002. Significantly more tissue damage (absorption) was visible under the wavelength grouping 455, 455 Y, 475, 475 Y, 495 and 515 than other combined wavelengths.

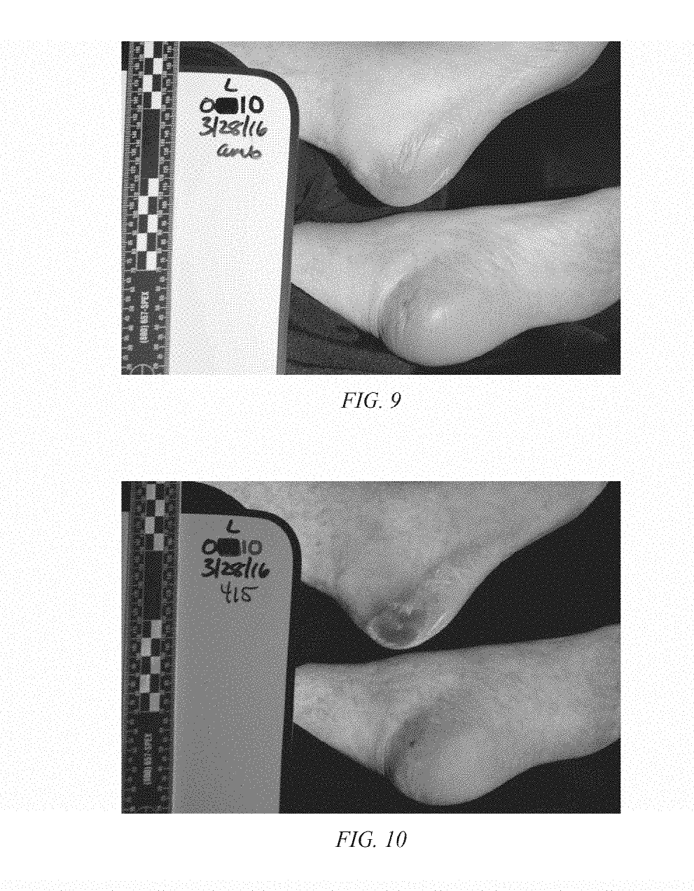

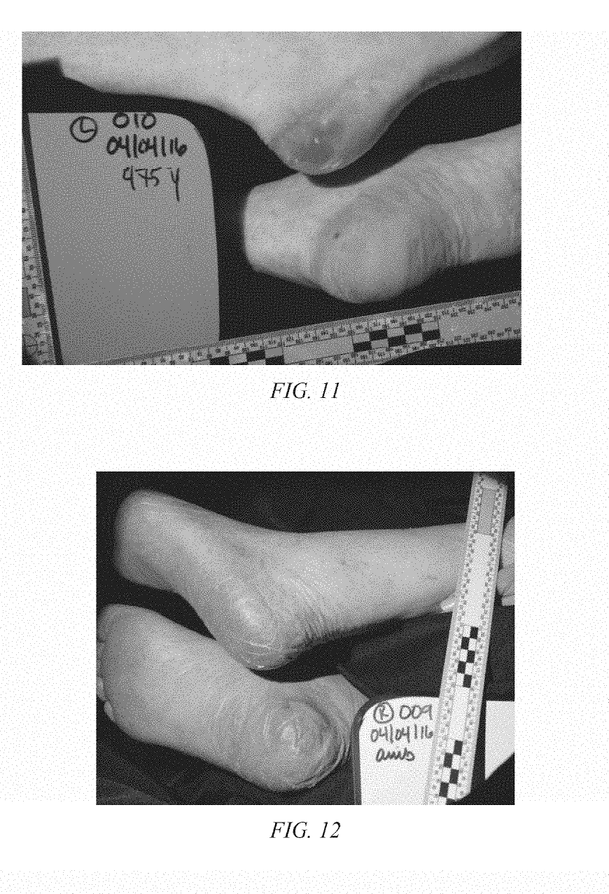

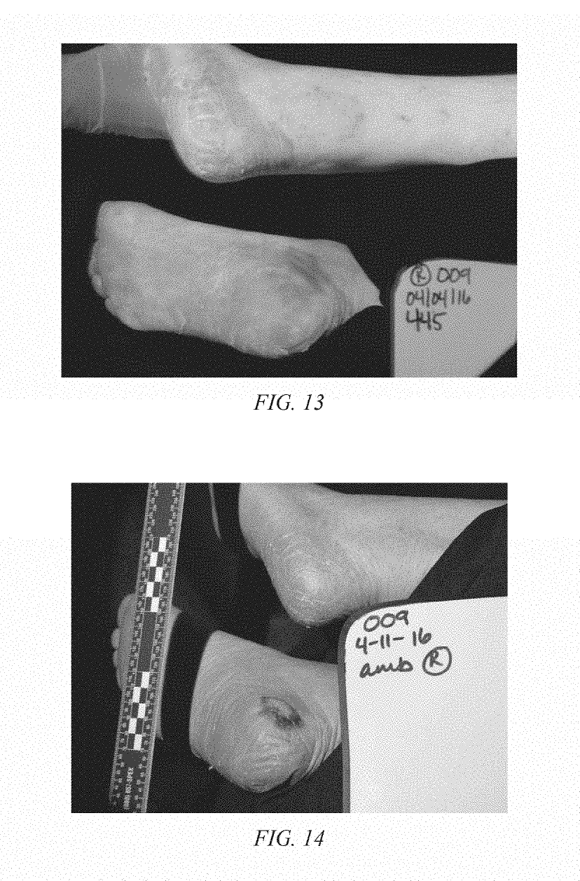

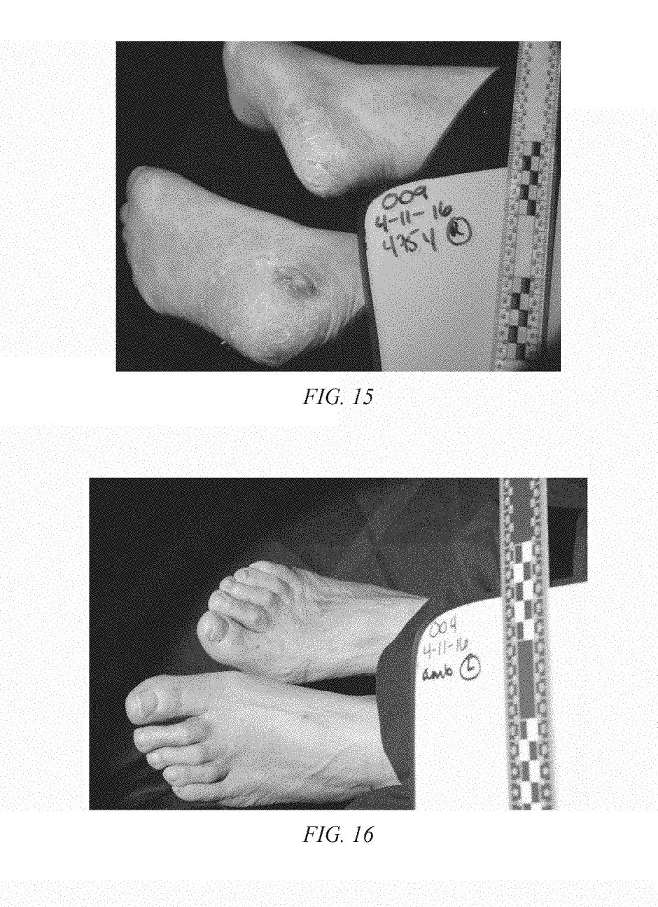

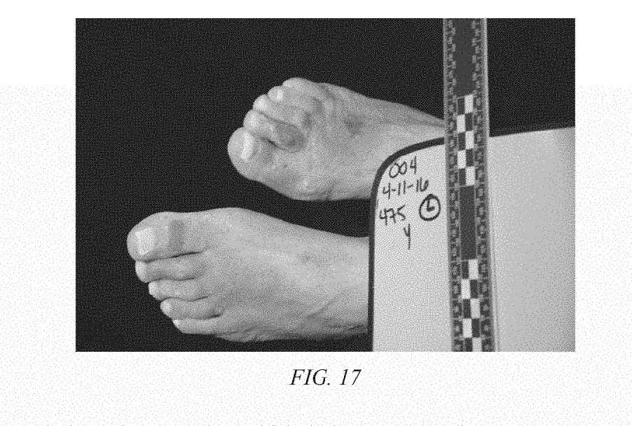

Subjects who presented with non-blanching erythema and existing ulceration in ambient light (Stage 1 pressure ulcer/injury; Unstageable pressure ulcer/injury) showed significant tissue absorption indicating the actual scope and magnitude of the tissue trauma under ALS. See FIGS. 9-15. Additionally, in the body region observed for this study, subjects with scars, areas of previous injury, and pigmentary changes also showed significant absorption at those sites beyond what was noted in ambient light. See FIGS. 16-17.

FIG. 8 shows a dendrogram, which is a visual representation of the image data. The images are arranged along the bottom of the dendrogram and referred to as leaf nodes. Clusters are formed by joining individual images or existing images clusters with the join point referred to as a node. At each dendrogram node we have a right and left sub-branch of clustered images. The vertical axis is labelled distance and refers to a distance measure between images or clusters. The height of the node can be thought of as the distance value between the right and left sub-branch clusters. The distance measure between two clusters is calculated as follows: D=1-C, where D=Distance and C=correlation between clusters.

If images are highly correlated, they will have a correlation value close to 1 and so D=1-C will have a value close to zero. Therefore, highly correlated clusters are nearer the bottom of the dendrogram. Clusters that are not correlated have a correlation value of zero and a corresponding distance value of 1. Clusters that are negatively correlated will have a correlation value of −1 and D=1−(−1)=2.

As we move up the dendrogram, the clusters get bigger and the distance between clusters increases in value. As such it becomes difficult to interpret distance between clusters when clusters increase in size. A possible way to think about the behavior of different images would be to see how far up the dendrogram you need to go so you can move between the images. In the dendrogram below, you see that to get from the first four images (L575-RA475) on the left to the images LA75 and RA75, you need to move up a distance of 0.8 (just follow the branches).

These combined findings indicate that ALS can detect tissue trauma not visible to the naked eye providing further details regarding extent and magnitude of tissue involvement. This non-invasive tool could help identify patients in the early stages of tissue trauma as well as screen for sites of previous injury that are at risk for subsequent breakdown.

TABLE 1
Descriptive Statistics

| Measures | Missing | No | Yes |
|---|---|---|---|
| Lamb | 3 (8.6%) | 23 (65.7%) | 9 (25.7%) |
| L.415 | 4 (11.4%) | 21 (60.0%) | 10 (28.6%) |
| L.445 | 4 (11.4%) | 21 (60.0%) | 10 (28.6%) |
| L.455 | 6 (17.1%) | 18 (51.4%) | 11 (31.4%) |
| L.455Y | 6 (17.1%) | 17 (48.6%) | 12 (34.3%) |
| L.475 | 6 (17.1%) | 20 (57.1%) | 9 (25.7%) |
| L.475Y | 11 (31.4%) | 13 (37.1%) | 11 (31.4%) |
| L.495 | 4 (11.4%) | 21 (60.0%) | 10 (28.6%) |
| L.515 | 4 (11.4%) | 23 (65.7%) | 8 (22.9%) |
| L.535 | 4 (11.4%) | 25 (71.4%) | 6 (17.1%) |
| L.555 | 5 (14.3%) | 25 (71.4%) | 5 (14.3%) |
| L.575 | 4 (11.4%) | 30 (85.7%) | 1 (2.9%) |
| L.575sp | 13 (37.1%) | 17 (48.6%) | 5 (14.3%) |
| R.amb | 3 (8.6%) | 19 (54.3%) | 13 (37.1%) |
| R.415 | 4 (11.4%) | 17 (48.6%) | 14 (40.0%) |
| R.445 | 4 (11.4%) | 17 (48.6%) | 14 (40.0%) |
| R.455 | 6 (17.1%) | 13 (37.1%) | 16 (45.7%) |
| R.455Y | 6 (17.1%) | 13 (37.1%) | 16 (45.7%) |
| R.475 | 6 (17.1%) | 16 (45.7%) | 13 (37.1%) |
| R.475Y | 11 (31.4%) | 13 (37.1%) | 11 (31.4%) |
| R.495 | 4 (11.4%) | 17 (48.6%) | 14 (40.0%) |
| R.515 | 4 (11.4%) | 17 (48.6%) | 14 (40.0%) |
| R.535 | 4 (11.4%) | 17 (48.6%) | 14 (40.0%) |
| R.555 | 5 (14.3%) | 17 (48.6%) | 13 (37.1%) |
| R.575 | 4 (11.4%) | 24 (68.6%) | 7 (20.0%) |
| R.575sp | 13 (37.1%) | 11 (31.4%) | 11 (31.4%) |

TABLE 2
Chi Square Analysis

| Measures | Missing | No | Yes |
|---|---|---|---|
| 455 Y and 475 Y | 90 (14.3%) | 362 (57.5%) | 178 (28.3%) |
| Other | 148 (16.3%) | 485 (53.3%) | 277 (30.4%) |
| 455, 455 Y, 475, 475 Y, | 74 (17.6%) | 201 (47.9%) | 145 (34.5%) |
| 495, and 515 | | | |
| Other | 164 (14.6%) | 646 (57.7%) | 310 (27.7%) |

The percentage of findings did not differ by grouping, c²(2, N = 1540) = 2.71, p = 0.257.
The percentage of findings did differ by grouping, c²(2, N = 1540) = 11.95, p = 0.002.
Significantly more “bruises” were found under the wavelength grouping 455, 455 Y, 475, 475 Y, 495, and 515, than other combined wavelengths.

### CONCLUSIONS

There are a number of factors associated with increased risk of pressure ulcer/injury development including black race, older age, poor nutritional status, lower body weight, physical or cognitive impairment, incontinence, and specific medical comorbidities that affect circulation such as diabetes or peripheral vascular disease (Moore, Z. et al. Cochrane Database of Systemic Reviews 2014). There are a number of instruments available to assess for the risk of pressure ulcers/injuries such as the Braden Scale, the Norton Scale, and the Waterlow Scale; however, there are no reliable tools to detect tissue trauma before visible manifestations are evident on the skin.

A study by Rowan et al. (J Forensic Legal Med 17(6):293-297 2010) conducted within the field of forensic medicine, may have relevant implications. Blunt force trauma may not show visual skin effects initially, but the subdermal breakdown may still be occurring. Rowan's study recognized that these changes beneath the skin may be revealed by other tools and looked into the detection of previous blunt force injury after the resolution of skin changes were no longer visible to the naked eye. Just as with pressure ulcers/injuries, many times the damage is not visible to the naked eye. In Rowan's study, investigators used an adapted digital camera and a standard Nikon camera, photographing ten volunteers over six months. There was no statistically significant difference between groups of bruises photographed with both the infrared digital camera that had been adapted to capture only infrared light compared to the standard camera which had the same lens fitted to it. The two groups were not significantly different with regard to what skin changes could be detected. The use of the near infrared spectrum, with wavelengths longer than the human eye can detect, did not reveal significant evidence of bruising after it had faded from view to both the naked eye and to a standard camera.

Also within the realm of forensic innovation in medicine is the study of ALS used in crime scene investigations to detect soft tissue injuries not seen under visible light. In a study done by Holbrook (Journal of Forensic Nursing 9(3): 140-145 2013), ALS was used to determine whether it was a valuable tool in visualizing acute trauma in cases of suspected strangulation. This study provides insight into the use of different wavelengths when utilizing ALS in order to detect injuries at various depths in the skin caused by acute trauma. In the methods of the study, different wavelengths are discussed as beneficial in visualizing a wounds' depth. For example, the study determined that most of the bruising caused by strangulation trauma was best seen using wavelengths 415-515 nanometers and multiple colored protection goggles. For subcutaneous wounds, the researchers recommend using infrared light to best visualize the wound because of the depth of tissue involved.

Holbrook concludes that ALS can aid in the examination and treatment of the victims of violence, which is different than this disclosure. Unlike bruising which is due to the rupture and spillage of blood into the perivascular tissues, pressure injuries involve tissue necrosis that manifest initially below the surface of the skin.

Because ALS utilizes different wavelengths than white light, it is able to reveal this tissue necrosis that would otherwise be invisible to the naked eye. As a skin and wound assessment tool, ALS appears to be beneficial in identifying early destructive tissue changes as well as sites of previous injury much sooner than just using white or ambient light and the naked eye. The ability to witness negative tissue changes before visual and physical manifestations on the skin appear is a breakthrough in pressure ulcer/injury detection.

All patents and publications mentioned in this specification are indicative of the levels of those skilled in the art to which the invention pertains. All patents and publications are herein incorporated by reference to the same extent as if each individual publication was specifically and individually indicated to be incorporated by reference. It is to be understood that while a certain form of the invention is illustrated, it is not intended to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein. The systems, methods, and devices described herein are presently representative of the preferred embodiments, are intended to be exemplary and are not intended as limitations on the scope. Changes therein and other uses will occur to those skilled in the art which are encompassed within the spirit of the invention. Although the invention has been described in connection with specific, preferred embodiments, it should be understood that the invention as ultimately claimed should not be unduly limited to such specific embodiments. Indeed various modifications of the described modes for carrying out the invention which are obvious to those skilled in the art are intended to be within the scope of the invention.

What is claimed is:

1. A system for detecting changes in tissue indicative of tissue damage prior to visibility of the changes on skin with an unaided eye, the system comprising:
an alternative light source (ALS) configured for emitting light in one or all of the ultraviolet, visible, and infrared wavelengths and for illuminating an area of tissue to be evaluated for changes;
a plurality of filters for a camera for obtaining images with different wavelengths of light emitted from the alternative light source; and
a camera configured for obtaining images of the area of tissue illuminated with the alternative light source.

2. The system according to claim 1, wherein the tissue damage results from at least one of mechanical deformation, pressure, shear, and friction applied to skin over time.

3. The system according to claim 1, wherein the alternative light source (ALS) is portable and handheld.

4. The system according to claim 3, wherein the alternative light source (ALS) is a forensic light source.

5. The system according to claim 4, wherein the alternative light source (ALS) is a SPEX Forensics Mini-CrimeScopet.

6. The system according to claim 5, wherein the SPEX Forensics Mini-CrimeScope® is usable at and switchable between 6, 8, 12, or 16 wavelengths of light.

7. The system according to claim 6, wherein the wavelength of light is selected from the group of frequencies consisting of 365 nm (ultraviolet), 390 nm, 415 nm, 445 nm, 455 nm, 475 nm, 495 nm, CSS SP (Short Pass) 540 nm, 515 nm, 535 nm, 555 nm, SP 575 nm, 575 nm, 600 nm, and white light.

8. The system according to claim 1, wherein the camera is affixed to the alternative light source (ALS).

9. The system according to claim 1, wherein the camera is an SLR (single-lens reflex) camera.

10. The system according to claim 1, wherein the plurality of filters includes a red camera lens, a yellow camera lens, and an orange camera lens.

11. The system according to claim 10, further comprising red goggles, yellow goggles, orange goggles, and black out goggles.

12. The system according to claim 11, further comprising a photographic grid for measurement and evaluation of the images.

13. The system according to claim 12, further comprising one or more of black sheets for providing contrast to the images and for reducing natural light when the system is in use and a tripod for supporting the camera.

14. A method for detecting tissue damage due to at least one of mechanical deformation, pressure, shear, and friction applied to skin over time, the method comprising:
illuminating skin using a forensic alternative light source (ALS) configured for examining crime evidence using a frequency of light selected for revealing tissue damage; and
observing the skin for indications of tissue damage visible only when the selected frequency of light is used.

15. The method according to claim 14, wherein the tissue damage is revealed by absorption of light.

16. The method according to claim 14, wherein the frequency of light is selected from the group consisting of a violet wavelength at about 415-445 nm, a blue wavelength at about 455-515 nm, and a green wavelength at about 535-575 nm.

17. The method according to claim 14, further comprising viewing the illuminated skin with a lens specific for selected wavelengths of light.

18. The method according to claim 17, further comprising at least one of viewing a violet wavelength using a yellow camera lens, viewing a blue wavelength using an orange camera lens, and viewing a green wavelength using a red camera lens.

19. The method according to claim 14, further comprising selecting an area of skin for illuminating.

20. The method according to claim 19, wherein the selecting includes selecting an area of skin overlying bone.

21. The method according to claim 20, wherein the selecting includes selecting an area of skin overlying a heel, a pelvis, a shoulder, a spine, a wrist, or an elbow of the patient.

22. The method according to claim 14, further comprising obtaining at least one image of the area of skin illuminated.

23. The method according to claim 22, further comprising obtaining at least one image of the area of skin in ambient light, prior to illuminating using the forensic alternative light source (ALS).

24. The method according to claim 14, further comprising obtaining at least one image of the area of skin in ambient light, prior to illuminating using the forensic alternative light source (ALS), followed by obtaining at least one image of the area of skin illuminated.

25. A method for detecting changes in tissue indicative of tissue damage prior to visibility of the changes on skin with an unaided eye, the method comprising:
selecting an area of skin of a subject to be evaluated for changes;
optimizing position of the subject for data collection;
establishing a baseline for the data collection by obtaining at least one image of the area of skin in ambient light using a camera;
reducing ambient light;
illuminating the area of skin using an alternative light source (ALS) configured for emitting light in one or all of the ultraviolet, visible, and infrared wavelengths;
obtaining a series of images of the area of skin illuminated by different wavelengths of light using the camera;
evaluating the images for changes in the tissue; and
documenting any changes observed in the tissue.

26. The method according to claim 25, wherein the tissue damage results from at least one of mechanical deformation, pressure, shear, and friction applied to skin over time.

27. The method according to claim 25, wherein the subject is a human patient.

28. The method according to claim 25, wherein optimizing the position includes positioning the subject lying on his/her side.

29. The method according to claim 25, further comprising, prior to illuminating the area of skin, pre-starting the alternative light source (ALS) and obtaining maximum luminosity.

30. The method according to claim 25, wherein the wavelengths of light are a violet wavelength at about 415-445 nm, a blue wavelength at about 455-515 nm, and a green wavelength at about 535-575 nm.

31. The method according to claim 25, further comprising viewing the illuminated skin with a lens specific for selected wavelengths of light.

32. The method according to claim 31, further comprising viewing a violet wavelength using a yellow camera lens, viewing a blue wavelength with an orange camera lens, and viewing a green wavelength using a red camera lens.

33. The method according to claim 25, further comprising viewing a violet spectrum at a wavelength of 415 nm and a wavelength of 445 nm using a yellow camera lens and yellow goggles, viewing a blue spectrum at a wavelengths of 455 nm, 475 nm, 495 nm, and 515 nm using an orange camera lens and orange goggles, and viewing a green spectrum at wavelengths of 535 nm, 555 nm, and 575 nm using a red camera lens and red goggles.

34. The method according to claim 33, further comprising viewing the blue spectrum at the wavelength of 455 nm and at the wavelength of 475 nm using the yellow camera lens and the yellow goggles.

35. The method according to claim 25, wherein obtaining a series of images includes taking a minimum of 12 photos of each subject.

36. The method according to claim 25, wherein the selecting includes selecting an area of skin overlying bone.

37. The method according to claim 36, wherein the selecting includes selecting an area of skin overlying a heel, a pelvis, a shoulder, a spine, a wrist, or an elbow of the subject.

38. The method according to claim 37, wherein the selecting includes selecting an area of skin overlying the heel of the subject.

39. The method according to claim 25, further comprising setting the F stop of the camera between 2.8 to 8.

40. The method according to claim 25, further comprising setting the exposure time of the camera at 1/100 of a second.

41. The method according to claim 25, further comprising placing the camera at a distance in a range of about 6 inches to about 24 inches from the subject.

42. The method according to claim 25, further comprising placing the camera at a distance of at least 24 inches from the subject.

43. The method according to claim 25, wherein evaluating the images includes observing an amount of absorption of light by the tissue.

44. The method according to claim 43, further comprising determining a relationship between the wavelength used and a number of tissue injuries detected.

45. The method according to claim 25, further comprising repeating the method for all subjects over a pre-determined period of time.

46. The method according to claim 45, wherein the method is repeated once a week for 6 consecutive weeks.

47. A system comprising:
an alternative light source (ALS) configured for emitting light in one or all of the ultraviolet, visible, and infrared wavelengths and for illuminating an area of tissue to be evaluated for changes;
a plurality of filters for a camera for obtaining images with different wavelengths of light emitted from the alternative light source; and
a camera configured for obtaining images of the area of tissue illuminated with the alternative light source;
for use in detecting changes in tissue indicative of tissue damage prior to visibility of the changes on skin with an unaided eye.

48. Use according to claim 47, wherein the tissue damage results from at least one of mechanical deformation, pressure, shear, and friction applied to skin over time.

49. Use according to claim 47, wherein the alternative light source (ALS) is portable and handheld.

50. Use according to claim 49, wherein the alternative light source (ALS) is a forensic light source.

51. Use according to claim 50, wherein the alternative light source (ALS) is a SPEX Forensics Mini-CrimeScope®.

52. Use according to claim 51, wherein the SPEX Forensics Mini-CrimeScope® is usable at and switchable between 6, 8, 12, or 16 wavelengths of light.

53. Use according to claim 52, wherein the wavelength of light is selected from the group of frequencies consisting of 365 nm (ultraviolet), 390 nm, 415 nm, 445 nm, 455 nm, 475 nm, 495 nm, CSS SP (Short Pass) 540 nm, 515 nm, 535 nm, 555 nm, SP 575 nm, 575 nm, 600 nm, and white light.

54. Use according to claim 47, wherein the camera is affixed to the alternative light source (ALS).

55. Use according to claim 47, wherein the camera is an SLR (single-lens reflex) camera.

56. Use according to claim 47, wherein the plurality of filters includes a red camera lens, a yellow camera lens, and an orange camera lens.

57. Use according to claim 56, further comprising red goggles, yellow goggles, orange goggles, and black out goggles.

58. Use according to claim 47, further comprising a photographic grid for measurement and evaluation of the images.

59. Use according to claim 58, further comprising one or more of black sheets for providing contrast to the images and for reducing natural light when the system is in use and a tripod for supporting the camera.
